(19) United States
(12) Patent Application Publication
(10) Pub. No.: US 2024/0392058 A1
(43) Pub. Date: Nov. 28, 2024

(54) ANTIMICROBIAL, AMPHIPHILIC COPOLYMER COATING FOR URINARY CATHETERS, CENTRAL LINE CATHETERS, AND OTHER DEVICES

(71) Applicants: Wan Y. SHIH, Bryn Mawr, PA (US); Wei-Heng SHIH, Bryn Mawr, PA (US); Jamie TRINH, Philadelphia, PA (US)

(72) Inventors: Wan Y. SHIH, Bryn Mawr, PA (US); Wei-Heng SHIH, Bryn Mawr, PA (US); Jamie TRINH, Philadelphia, PA (US)

(73) Assignee: Drexel University, Philadelphia, PA (US)

(21) Appl. No.: 18/671,495

(22) Filed: May 22, 2024

Related U.S. Application Data
(60) Provisional application No. 63/504,211, filed on May 25, 2023.

(51) Int. Cl. C08F 299/02; A61L 29/08; A61L 29/16

(52) U.S. Cl. CPC C08F 299/02; A61L 29/085; A61L 29/16; A61L 2300/404; A61L 2420/02; C08F 2810/20

(57) ABSTRACT

An antimicrobial amphiphilic copolymer comprising units derived from one or more hydrophobic polymers and one or more hydrophilic polymers, wherein the copolymer is selected from the group consisting of non-ionic and anionic copolymers and the copolymer is a random copolymer or a block copolymer, methods for making the copolymer, coatings made from the copolymer and medical devices coated with the copolymer.

### CROSS REFERENCE TO RELATED APPLICATIONS

This application claims the benefit of U.S. Provisional Application No. 63/504,211 filed on May 25, 2023, the disclosure of which is hereby incorporated by reference in its entirety as if set forth fully herein.

### STATEMENT OF GOVERNMENT INTEREST

This invention was made with government support under contract Nos. 1R43AI152716-01 and R03EB030215 awarded by the National Institutes of Health, and contract No. 58-8072-9-024 awarded by the U.S. Department of Agriculture. The government has certain rights in the invention.

### BACKGROUND

Approximately 5 million central venous catheters (CVC) are inserted every year in the US alone [1, 2], with up to 8% leading to central line-associated bloodstream infection (CLABSI) [3]. CLABSI is one of the most expensive healthcare infections. Each CLABSI costs approximately an additional US$28,000 per patient. The burden of CLABSI is over US$2 billion a year in the US alone [4].

Urinary-tract catheters are the second most inserted medical device. Catheter-associated urinary tract infection (CAUTI) is one of the most prominent health-associated infections (HAIs) and the second most common infection affecting the geriatric population [5]. CAUTI can be caused by extended use of the urinary-tract catheters, improper techniques, or unsterile equipment used when inserting the catheter. 75% of hospital patients receiving a urinary-tract catheter will contract a urinary-tract infection [6], costing an additional US$2,800 per patient [7, 8]. The burden of CAUTI is US$830 million a year in the US alone [6].

There is a need to prevent or reduce central-line and urinary catheter-associated infections. The cause of these catheter-associated infections is related to the presence of bacteria and their ability to attach to and colonize the catheter surface [6]. A long-lasting antimicrobial coating that can prevent bacterial attachment and colonization on catheter surfaces would be ideal for preventing catheter-associated infections.

Existing surface modification strategies include anti-adhesive or bactericidal coatings. Although anti-adhesive coatings can prevent protein and bacterial attachment, they fail to affect bacterial viability [9]. One example of an anti-adhesive coating is poly(ethylene glycol) (PEG) [10], a nonionic, hydrophilic polymer. Although it can prevent bacterial adhesion, it has the potential to form aldehydes under physiological conditions which are toxic to the body, and thus undesirable.

Silver antimicrobial agents or antibiotics embedded in coatings deplete over time [10], making their efficacy temporary. So far, there has been no good way to slow the release of these compounds over time, thus making them ineffective for extended use [11]. For example, bactericidal coatings such as the silver-alloy hydrogel coating [12-15] offers good initial antimicrobial performance but displays limited antimicrobial action over time, limiting its utility to less than one week. It also offers minimal prevention against bacterial attachment in vivo [12-15]. The use of silver nanoparticles in central line surface coatings can compromise blood compatibility and lead to catheter-associated thrombus formation [16]. In addition to these disadvantages, silver-containing antimicrobial coatings also significantly stiffen the catheters, making them harder to insert without causing blood vessel injuries.

Another aspect of typical bactericidal coatings is that they contain positive charges or are hydrophobic in nature, both of which can increase bacterial attachment to the surface. Quaternary ammonium silane coatings are an example of coatings containing positive charges, which typically fail to prevent bacterial attachment due to the positive charge of the immobilized compound which attracts bacteria to the surface [18-25]. Examples of hydrophobic bactericidal agents that can also increase bacterial adsorption on the surface can be found in Refs. [26, 27]. Although these coatings can inhibit the initial bacterial attachment to the coating, inhibited bacteria and their debris could still attach to the surface over time, effectively forming a protective layer that diminishes the effect of the bactericidal coating and promoting the growth of biofilms [6, 28].

Mushtaq, Shehla, et al. describes amphiphilic copolymers for use as antibacterial agents. The amphiphilic copolymers of Mushtaq et al. comprise copolymers of dimethylamino ethyl methacrylate and methyl methacrylate to control biofilm adhesion for antifouling applications [36].

Chang, Chih-Hao, et al. relates to a polystyrene-block-quaternized polyisoprene (PS-b-PIN) amphipathic block copolymer obtained by anionic polymerization. The PS-b-PIN is reacted with N,N-dimethyldodecylamine as a side chain to form a polymersome structure. [37].

Therefore, there is a need for an antimicrobial coating that not only inhibits bacteria but also prevents bacterial attachment to provide a long-lasting antimicrobial effect that is not cytotoxic and is soft and durable to minimize potential tissue and blood vessel injuries during intubation.

### SUMMARY

The present invention may be described by the following sentences:
1. In a first aspect, the present invention may relate to an antimicrobial amphiphilic copolymer comprising one or more hydrophobic polymers and one or more hydrophilic polymers,
   wherein the copolymer is non-ionic or anionic and the copolymer is a random copolymer or a block copolymer.
2. The antimicrobial amphiphilic copolymer of sentence 1, wherein the hydrophobic polymer may be selected from the group consisting of poly-vinyl acetate (PVAc) and poly(maleic anhydride-alt-1-octadecene) (PMAO); and
   the hydrophilic polymer may be selected from a group that is negatively-charged or with no charges including, optionally, the hydrophilic polymer may be selected from the group consisting of poly-(methyl vinyl ether)-maleic anhydride (PMVE-MA), polyvinyl-alcohol (PVA), polyethylene glycol (PEG), or any combination of PMVE-MA, PVA and PEG
3. The antimicrobial amphiphilic copolymer of any one of sentences 1-2, wherein the hydrophobic polymer may have a molecular weight of from about 20 D to about 10,000,000 D, or from about 50,000 D to about 400,000 D, or from about 75,000 D to about 350,000 D, as measured by gel permeation chromatography.
4. The antimicrobial amphiphilic copolymer of any one of sentences 1-3, wherein the hydrophilic polymer may have a molecular weight of from about 20 D to about 10,000,000 D, or from about 50,000 D to about 400,000 D, or from about 75,000 D to about 350,000 D, as measured by gel permeation chromatography.
5. The antimicrobial amphiphilic copolymer of any one of sentences 1-4, wherein copolymer may have a molar ratio of the hydrophobic polymer to the hydrophilic polymer of from about 0.0001 to 10000.
6. The antimicrobial amphiphilic copolymer of any one of sentences 1-5, wherein the hydrophobic polymer may be poly-vinyl acetate (PVAc) and the hydrophilic polymer may be polyethylene glycol (PEG).
7. The antimicrobial amphiphilic copolymer of any one of sentences 1-5, wherein the hydrophobic polymer may be poly-vinyl acetate (PVAc) and the hydrophilic polymer may be polyvinyl-alcohol (PVA).
8. The antimicrobial amphiphilic copolymer of any one of sentences 1-5, wherein the hydrophobic polymer may be poly-vinyl acetate (PVAc) and the hydrophilic polymer may be poly-(methyl vinyl ether)-maleic anhydride (PMVE-MA).
9. The antimicrobial amphiphilic copolymer of any one of sentences 1-5, wherein the hydrophobic polymer may be poly(maleic anhydride-alt-1-octadecene) (PMAO) and the hydrophilic polymer may be polyethylene glycol (PEG).
10. The antimicrobial amphiphilic copolymer of any one of sentences 1-5, wherein the hydrophilic polymer may be a hydroxyl group containing hydrophilic polymer.
11. The antimicrobial amphiphilic copolymer of sentence 10, wherein the hydrophilic polymer is selected from the group consisting of poly(vinyl alcohol), glycerol propoxylate, poly(vinyl alcohol co-ethylene, poly(2-hydroxyethyl methacrylate), α,ω-bis(hydroxy)-terminated poly(ethylene glycol), α,ω-bis(hydroxy)-terminated poly(ethylene oxide), α-dibenzylmethylene-terminated pooly(ethylene glycol), poly(ethylene glycol) decyl ether, poly(ethylene glycol)ethyl ether, poly(ethylene glycol) and mixtures of two or more of these hydrophilic polymers.
12. The antimicrobial amphiphilic copolymer of any one of sentences 1-5, wherein the hydrophobic polymer may be a carboxyl group containing hydrophobic polymer.
13. The antimicrobial amphiphilic copolymer of sentence 12, wherein the hydrophilic polymer is selected from the group consisting of poly(acrylic acid), poly(ethylene-alt-maleic anhydride), poly(4-styrenesulfonic acid-co-maleic acid), poly(methyl vinyl ether-alt-maleic anhydride), poly(methyl vinyl ether-alt-maleic acid, poly(styrene-alt-maleic acid), poly(methyl vinyl ether-alt-maleic acid monoethyl ester), poly(2-propylacrylic acid), poly(2-ethlacrylic acid), poly(α-ethylacrylic acid), poly(methacrylic acid), poly(α-propylacrylic acid) and mixtures of two or more of these hydrophilic polymers.
14. The antimicrobial amphiphilic copolymer of any one of sentences 1-5 and 10-13, wherein the hydrophobic polymer is selected from the group consisting of poly(vinyl acetate), poly(ethylene-vinyl acetate) and mixtures thereof.
15. The antimicrobial amphiphilic copolymer of any one of sentences 1-14, wherein the copolymer may be devoid of amine functionalities or any positively charged functionalities.
16. The antimicrobial amphiphilic copolymer of any one of sentences 1-15, wherein the minimum inhibitory concentration (MIC) of the copolymers for SA and MRSA may be from about 0.1 μg/ml-1000 μg/ml, or from about 0.01 μg/ml to about 50 μg/ml, or from about 0.1 μg/ml to about 32 μg/ml, as determined by a broth microdilution assay.
17. The antimicrobial amphiphilic copolymers of any one of sentences 1-16, wherein the copolymer may inhibit both Gram-positive and Gram-negative bacteria with an MIC in the range of from about 0.01 μg/ml-1000 μg/ml, or from about 0.01 μg/ml to about 50 μg/ml, or from about 0.1 μg/ml to about 32 μg/ml, as determined by a broth microdilution assay.
18. The antimicrobial amphiphilic copolymers of any one of sentences 1-16, wherein the copolymer may inhibit both antibiotic-susceptible and antibiotic-resistant bacteria with an MIC in the range of from about 0.01 μg/ml-1000 μg/ml, or from about 0.01 μg/ml to about 50 μg/ml, or from about 0.1 μg/ml to about 32 μg/ml, as determined by a broth microdilution assay.
19. In a second aspect, the present invention relates to a method for preparing the antimicrobial amphiphilic copolymer of any one of sentences 6-8, comprising a step of partially hydrolyzing a PVAc polymer using an acid-catalyzed hydrolysis reaction to form a poly-vinyl acetate-polyvinyl-alcohol (PVAc/PVA) random copolymer comprising a vinyl acetate—vinyl alcohol (Vac/VA) segment of the copolymer according to Formula I:

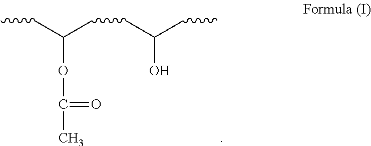

20. In a third aspect, the present invention relates to a method of preparing the antimicrobial amphiphilic copolymer of sentence 8, comprising:
   partially hydrolyzing a PVAc polymer using an acid-catalyzed hydrolysis reaction to form a poly-vinyl acetate-polyvinyl-alcohol (PVAc/PVA) random copolymer, and
   reacting the poly-vinyl acetate-polyvinyl-alcohol random copolymer with poly(methyl vinyl ether-co-maleic acid) (PMVE-MA) via a condensation reaction to form a PVAc/PMVE-MA copolymer comprising a vinyl acetate-methyl vinyl ether-co-maleic acid (Vac/MVE-MA) segment of the copolymer according to Formula II:

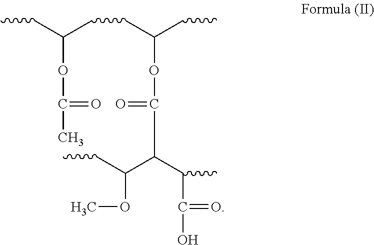

21. In a fourth aspect, the present invention relates to a method of preparing the antimicrobial amphiphilic copolymer of sentence 9, comprising reacting poly(maleic anhydride-alt-1-octadecene) and polyethylene via a condensation reaction to form a poly(maleic anhydride-alt-1-octadecene)-polyethylene glycol (PMAO/PEG) random copolymer.
22. In a fifth aspect, the present invention relates to a method of preparing the antimicrobial amphiphilic copolymer of sentences 1-6, comprising:
   partially hydrolyzing a PVAc polymer using an acid-catalyzed hydrolysis reaction to form a poly-vinyl acetate-polyvinyl-alcohol (PVAc/PVA) random copolymer, and
   reacting the polyvinyl acetate-polyvinyl-alcohol random copolymer with PEG to form polyvinyl acetate-polyethylene glycol (PVAc/PEG) random copolymer.
23. In a sixth aspect, the present invention relates to an antimicrobial amphiphilic copolymer coating consisting of a monolayer coating comprising the antimicrobial amphiphilic copolymer of any one of sentences 1-22, wherein the hydrophobic component of the copolymer facilitates binding of the copolymer to a surface and the hydrophilic part forms an outer surface, and optionally, the coating is a thin, smooth, and durable monolayer coating on a medical device surface.
24 The coating of sentence 23, wherein the coating may be coated on the surface of a catheter, such as a urinary tract catheter, a central-line catheter, an intravenous catheter or other devices.
25. The coating of any one of sentences 23-24, wherein the coating when applied to a hydrophobic surface, such as a medical device surface, may be configured to modify a wetting angle of the hydrophobic surface from above 110° to below 40°, or from above 90° to below 80°, or from above 80° to below 80°, or from above 70° to below 70° and wherein the hydrophobic surface may be a silicone surface, a polyurethane surface, or a metal surface.
26. The coating of any one of sentences 23-25, wherein the coating may last for more than 30 days, or more than 60 days, when exposed to water, saline solution, broth, urine or serum, while providing protection against bacterial attachment and growth.
27. The coating of any one of sentences 23-26, wherein the coating may be applied to one or both of an interior or an exterior surface of a medical device surface.
28. The coating of any one of sentences 23-27, wherein the coating may be thin, for example, the coating may have a thickness of from about 1 nm to 10, 0000 nm or from 10 nm to 10,000 nm, or from 100 nm to 10,000 nm, or from 1000 nm to 10,000 nm.
29. The coating of any one of sentences 23-28, wherein the coating may be nontoxic to human cells in a MIC concentration range of the copolymers of from about 0.0001 μg/ml-10000 μg/ml, or from about 0.001 μg/ml to about 50 μg/ml, or from about 0.1 μg/ml to about 32 μg/ml, as determined by a broth microdilution assay.

### BRIEF DESCRIPTION OF THE DRAWINGS

FIG. 1A shows a schematic of an amphiphilic, monolayer copolymer coating on a silicone catheter surface where the hydrophobic component (black outlined line) sticks to the catheter surface and the hydrophilic component (solid black line) extends to the liquid environment.

FIG. 1B shows a schematic of a hydrolysis reaction for polyvinyl acetate (PVAc) that can lead to partial hydrolysis of PVAc depending on the conditions and duration of the hydrolysis.

FIG. 1C shows a schematic of crosslinking between PVAc/poly(vinyl alcohol) (PVA) and poly(methyl vinyl ether-co-maleic acid) (PMVE-MA) that can lead to the formation of a random amphiphilic copolymer PVAc/PMVE-MA.

FIG. 2A shows Fourier transform infrared spectra (FTIR) of PVAc/PVA copolymers with various theoretical vinyl alcohol (VA) concentrations as calculated based on Eq. (3). The vertical dashed line indicates the alcohol (—OH) peak position at 3330 cm⁻¹.

FIG. 2B shows the alcohol (—OH) peak intensity at around 3330 cm⁻¹ versus VA concentration. The straight line is the least square fit to the data points. The alcohol (—OH) peak at around 3330 cm⁻¹—a signature of VA—increased linearly with an increasing VA concentration, suggesting that the reactions were robust. Since the VA concentration was not verified, the theoretical VA concentration as calculated using Eq. (3) was therefore not a quantitative gauge of the VA concentration but a qualitative estimate of the extent of the conversion of VAc to VA.

FIG. 3 shows optical micrographs of a 1 ml deionized water droplet on each of (a) an uncoated silicone surface, (b) a PVAc/PVA30 (30 mM VA concentration) coated silicone surface, and (c) a PVAc/PMVE-MA-coated silicone surface. As can be seen, the coatings of PVAc/PVA30 and PVAc/PMVE-MA on a silicone surface reduced the wetting angles from 110° to 60° and 110° to 40°, respectively.

FIG. 4 shows the wetting angle of soaked (full circles) and unsoaked (open squares) of (a) PVAC/PVA40-coated silicone surfaces and (b) uncoated silicone surfaces. The soaked (full circles) and unsoaked (open squares) uncoated silicone surfaces remained at 110°. The wetting angle of the PVAc/PVA40-coated silicone surface remained at 70° throughout the 30 days indicating the durability of the PVAc/PVA coating in water.

FIG. 5A shows the relative bacterial viability as measured by the optical density of methicillin-resistant Staphylococcus aureus (MRSA) and Staphylococcus aureus (SA) treated with various concentrations of PVAc/PVA. PVAc/PVA exhibited μg/ml minimum inhibitory concentrations (MIC's) of: 16 μg/ml and 32 μg/ml of PVAc/PVA for SA (*) and MRSA (**), respectively.

FIG. 5B shows the relative bacterial viability as measured by the optical density of MRSA and SA treated with various concentrations of PVAc/PMVE-MA. PVAc/PMVE-MA exhibited a μg/ml MIC of: 8 μg/ml and 16 μg/ml of PVAc/PVA for SA (*) and MRSA (**), respectively,

FIG. 5C shows the relative bacterial viability as measured by the optical density of MRSA and SA treated with various concentrations of PMVE-MA. PMVE-MA alone exhibited an MIC exceeding 5000 μg/mL for both SA (*) and MRSA (**).

FIG. 6 shows the percent bacterial viability of (a) Pseudomonas aeruginosa (PA) (ATCC27853) culture incubated with PVAc/PMVE-MA copolymer at various concentrations. As can be seen in FIG. 6, the % Bacterial viability of PA diminished at 2 μg/mL of PVAc/PMVE-MA, indicating the MIC of PVAc/PMVE-MA to PA to be 2 μg/ml, which is comparable to the MIC's of antibiotics. Statistics were based on Welch's t-test with the significance level α=0.05: *** p=<0.03, **** p=<0.005, and n.s.=not significant.

FIG. 7 shows the % Bacterial viability of Escherichia coli (EC) K12 (ATCC 10798) culture incubated with PVAc/PMVE-MA copolymer at various concentrations. As can be seen, the % Bacterial viability of E. coli K12 diminished at 1 μg/mL, indicating the MIC of PVAc/PMVE-MA to E. coli K12 to be 1 μg/ml. which is comparable to the MIC's of antibiotics. Statistics were based on Welch's t-test with the significance level α=0.05: * p=0.0016, ** p<0.002, and n.s.=not significant.

FIG. 8 shows normalized-to-uncoated % bacterial viability of EC K12 in saline on the PVAc/PVA60-coated, PVAc/PVA50-coated, and PVAc/PVA30-coated at 23%, 21%, and 6.5%, respectively. Statistics were based on Welch's t-test with the significance level α=0.05: * p=0.01, ** p=0.001, *** p=0.004, and n.s.=not significant.

FIG. 9 shows normalized-to-uncoated % bacterial viability of EC K12 in saline on the PVAc/PVA30-coated, and PVAc/PMVE_MA-coated silicone surfaces. The normalized-to-uncoated % bacterial viability was 7.4%, and 2.8% for PVAc/PVA30-coated, and PVAc/PMVE-MA-coated silicone surfaces, respectively. Based on statistical testing, there was no difference between the % bacterial viability of PVAc/PVA30 and PVAc/PMVE-MA. This indicates that PVAc/PVA30 possesses a similar antimicrobial effect as PVAc/PMVE-MA which contains a known antimicrobial agent, PMVE-MA. Statistics were based on Welch's t-test with the significance level α=0.05: * p=<0.001, and n.s.=not significant.

FIG. 10 shows normalized-to-uncoated % bacterial viability of PA (ATCC 27853) in broth on a PVAc/PMVE-MA-coated silicone surface, which shows the normalized-to-uncoated bacterial viability on PVAc/PMVE-MA coating was 4%±1%, which was within the experimental uncertainty in saline solution. Statistics were based on Welch's t-test with the significance level α=0.05: * p=0.00008.

FIG. 11 shows normalized-to-uncoated % bacterial viability of SA (ATCC 29213) in broth on PVAc/PMVE-MA-coated silicone. The bacterial viability on PVAc/PMVE-MA-coated silicone was 6%±2%, which is also within the experimental uncertainty in saline solution. Statistics were based on Welch's t-test with the significance level α=0.05: * p=0.01.

FIG. 12 shows a comparison of % bacterial viability of MRSA (ATCC baa-1026) on a PVAc/PMVE-MA coated surface to % bacterial viability on an uncoated silicone surface. The PVAc/PMVE-MA coating reduced the % bacterial viability of MRSA by 88% (n=8), comparable to the 94% reduction of % bacterial viability of SA (ATCC 29213) by the PVAc/PMVE-MA coating, indicating that the PVAc/PMVE-MA coating was similarly effective in inhibiting the multi-antibiotic-resistant MRSA as the antibiotic-susceptible SA. Statistics were based on Welch's t-test with the significance level α=0.05: **** p=0.00002.

FIG. 13 is a summary of normalized-to-uncoated % bacterial viability of MRSA (ATCCbaa1026), SA (ATCC29213) and PA (ATCC27853) in broth and of EC (ATCCK12) in saline on uncoated and PVAc/PMVE-MA-coated silicone surfaces. This is a replot combining the data of FIGS. 9-12. PVAc/PMVE-MA-coated silicones significantly lowered % bacterial viability of SA and PA suspended in broth and EC K12 suspended in saline in comparison to uncoated silicone by 94%, 96% and 97%, respectively. Based on statistical testing using Welch's t-test with a significance level α=0.05, there was no significant difference between the % bacterial viability of bacteria suspended in broth or in saline caused by the PVAc/PMVE-MA-coated silicone surface. FIG. 13 clearly illustrates that the PVAc/PMVE-MA coating is capable of effectively inhibiting both Gram-negative bacteria (EC and PA) and Gram-positive bacteria (SA and MRSA) whether they were in saline solution or in broth. Importantly, this coating could inhibit both MRSA and SA by about 90%. This indicates that the coating was equally effective in inhibiting both antibiotic susceptible and antibiotic-resistant bacteria, a clear advantage in combating antibiotic resistance. * p<0.00008, ** p<0.01, *** p=0.005, **** p=0.00002.

### DETAILED DESCRIPTION OF THE INVENTION

The present invention offers an effective antimicrobial coating for urinary tract, central-line catheters, or other devices using amphiphilic copolymers to prevent infections. The coating is effective in inhibiting bacteria, reducing their viability by 90% regardless of whether the bacteria are Gram-positive or Gram-negative or if the bacteria are antibiotic-susceptible or antibiotic-resistant. The coating is also soft, non-abrasive, and durable.

A first aspect of the invention is the creation of antimicrobial amphiphilic copolymers that contain no known active antimicrobial functionalities but are highly effective in inhibiting bacteria with a minimum inhibitory concentration (MIC) in the μg/ml range, 1000-fold lower than that of a typical antimicrobial polymer containing known antimicrobial functionalities. For example, the amphiphilic copolymer and coating comprising the amphiphilic copolymer of the present invention contains no active antimicrobial agents, such as positively charged amine groups. As such, the present invention may be described as devoid of side chain amine functionalities. As a result, the amphiphilic copolymers of the present invention are non-ionic, negatively charged, or anionic. The copolymers of the present invention are advantageous relative to other amphiphilic copolymers since they are devoid of any positive charges, such as positively-charged amine functionalities, which are known to be cytotoxic as they disrupt cellular membranes.

This is advantageous since these properties minimize the binding strength of the coating to a cell surface. This, in turn, reduces scratching or pulling of cells from tissue by the coated object thereby reducing possible injury to tissue, or blood vessels during catheterization and/or intubation. Thus, objects coated with the coating of the present invention are less likely to disrupt the cellular membrane to cause injury, when compared to polymers containing positively charged amino groups.

The antimicrobial effect of the current antimicrobial amphiphilic copolymers is primarily due to the amphiphilicity of the copolymer and its high molecular weight. This makes the antimicrobial amphiphilic copolymers of the present invention different from antimicrobial polymers in current use, whose antimicrobial properties come primarily from the presence of known active antimicrobial functionalities therein. Additionally, the present amphiphilic copolymers are noncytotoxic to human cells.

A second aspect of the present invention relates to the creation of a thin, soft, and amphiphilic coating for, for example, a catheter surface. A catheter's surface is typically hydrophobic. The amphiphilic coating of the present invention, when applied to a catheter creates a hydrophilic catheter surface. The coating is strong and durable and provides long-lasting antimicrobial protection because the antimicrobial property of the coating comes from the copolymer rather than reactive groups in the copolymer. As such, there is no finite amount of an antimicrobial drug to elute from the coating.

The hydrophilic outer layer of the coating of the present invention makes the coating extremely smooth and non-abrading which can minimize potential catheter-inflicted injuries to tissue and blood vessels during intubation.

The goal of the present invention is to create an antimicrobial coating that is highly effective and long lasting to combat catheter-associated infections.

The present invention relates to: (1) preparing amphiphilic copolymers free from active antimicrobial agents or positive charges but which are highly antimicrobial, and (2) coating a surface of a catheter or other device with a monolayer of the amphiphilic copolymer for effective and long-lasting infection protection. The coating is a monolayer due to the amphiphilicity of the copolymer.

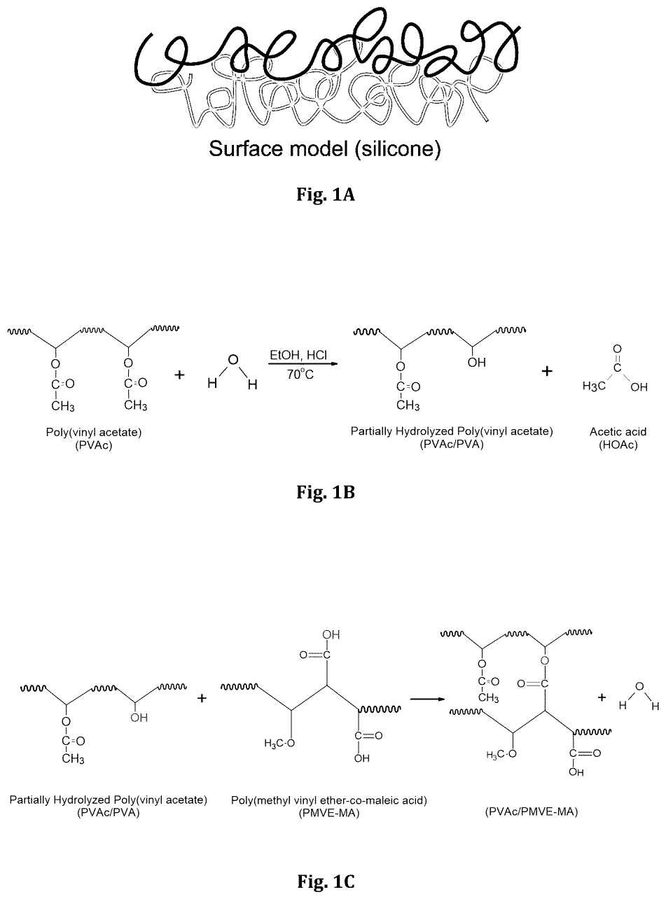

An amphiphilic copolymer comprises a hydrophobic component and a hydrophilic component. For example, the amphiphilic copolymer of the present invention may include a hydrophobic component of the copolymer which adheres to a surface, such as a catheter surface, to form the “inside” of the coating and a hydrophilic component that extends into the aqueous environment, to form the hydrophilic “outside” of the coating. (See the schematic shown in FIG. 1A).

The binding of the amphiphilic copolymer monolayer is strong and durable since each hydrophilic component and each hydrophobic component of the copolymer have many repeating units capable of binding, thereby fortifying the binding of the entire copolymer on a surface. The strong binding of the copolymer on the catheter surface contributes to the durability of the coating and its antimicrobial properties.

In addition to the durable antimicrobial properties, the amphiphilic nature of the copolymer coating also makes it thin, soft, and smooth, causing minimal friction and thus, reducing injuries to tissue and blood vessels.

Furthermore, unlike existing antimicrobial polymers that contain known active antimicrobial agents such as positively charged amine groups, the antimicrobial amphiphilic copolymers in this invention are non-ionic, negatively charged, or anionic, minimizing the binding strength of the coating to the cell surface. This minimizes scratching or pulling out of cells from tissue, which reduces possible injury to tissue, or blood vessels during catheterization and/or intubation.

### EXAMPLES

### Example 1

### Preparation of a Poly(Vinyl Acetate)/Poly(Vinyl Alcohol) (PVAc/PVA) Amphiphilic Copolymer

A PVAc/PVA amphiphilic copolymer consists of PVAc and PVA as the hydrophobic and hydrophilic components of the copolymer, respectively.

### Synthesis of the PVAc/PVA Amphiphilic Copolymers

A random PVAc/PVA amphiphilic copolymer was made by partially hydrolyzing PVAc as shown schematically in FIG. 1B using an acid-catalyzed hydrolysis reaction in a mixture of ethanol (Fisher Scientific), deionized (DI) water, and 37% hydrochloric acid (HCl) (Sigma) in a molar ratio of 87:34:1 (relative to 37% HCl) and a varied amount of acetic acid (MW 60.05, Fisher Scientific) to achieve different PVA/PVAc molar ratios in the copolymers. The reaction was carried out with 1 wt. % of PVAc by placing 1 g of PVAc beads (MW 100,000D, Fischer) in 99 g of the mixture of ethanol, DI water, hydrochloric acid and acetic acid, located in a flask connected to a condenser for reflux and kept in a 70±2° C. water bath and under constant stirring for up to 5 hr before being removed from the water bath to cool, and store at room temperature.

### PVAc/PVA Composition Control

The formation of a random PVAc/PVA copolymer was achieved using a hydrolysis reaction to convert a vinyl acetate (VAc) monomer, . . . . C₄H₆O₂ . . . , within the PVAc polymer to a vinyl alcohol (VA) monomer, . . . . C₂H₄O . . . as follows:

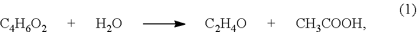

where H₂O is water, and CH₃COOH is acetic acid (HOAc), respectively. The chemical structures in Eqn. 1 can be seen in the schematic in FIG. 1B. For brevity Eq. (1) can be rewritten as:

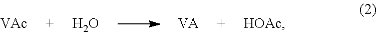

The equilibrium molar concentrations of reactants in Eq. (2) can be described as follows [30]:

$${K}_{eq} = \frac{[ VA ] [ HOAc ]}{[ VAc ] [ {H}_{2} O ]} \qquad ( 3 )$$

where [VA], [HOAc], [VAc], and [H₂O] are the equilibrium molar concentrations of the VA monomers, acetic acid, VAc monomers, and water, respectively. Assuming the Gibbs free energy difference of the reaction, ΔG°, is independent of temperature, the equilibrium constant at the reaction temperature, 70° C. (343.15 K), was estimated as K_{eq}=0.77 based on K_{eq}=0.75, at 35° C. (308.15 K) as reported in Ref. [30].

After the reaction, the reaction mixture was cooled and stored at room temperature before use. Considering the current reactions were conducted at a low initial weight percent of 1 wt. % of PVAc and both H₂O and HOAc were present at a much higher molar concentration, it was assumed that the molar concentrations of H₂O and HOAc would not change significantly during the reaction. Hence, the initial concentrations of H₂O and HOAc were used to estimate the [VA]/[VAc] in Eq. (3). 1 wt. % of PVAc was equivalent to 100 mM of VAc monomer and since [VAc]+[VA]=100 mM, it followed that a final [VA] could be deduced from Eq. (3) given a set of [H₂O] and [HOAc] concentrations. Thus, a final equilibrium VA molar concentration could be achieved primarily by controlling the amount of the added acetic acid.

In Table 1, the molar concentrations of acetic acid and water needed for various final molar concentrations of VA are listed. The reaction depicted in Eq. (3) is for the conversion of only some of the VAc monomers to VA within a PVAc polymer to form a PVAc/PVA random copolymer. Thus, the reaction only changes the VAc/VA composition within the polymer but does not change the overall polymer length or the polymer molar concentration.

TABLE 1

| | [H₂O] | [HOAc] | [VA] |
|---|---|---|---|
| | (M) | (M) | (mM) |
| PVAc/PVA10 | 2.3 | 15.6 | 10 |
| PVAc/PVA20 | 4.7 | 14 | 20 |
| PVAc/PVA30 | 7 | 12.3 | 30 |
| PVAc/PVA40 | 9.4 | 10.6 | 40 |
| PVAc/PVA50 | 11.8 | 8.9 | 50 |
| PVAc/PVA60 | 14.2 | 7.1 | 60 |
| PVAc/PVA70 | 16.7 | 5.4 | 70 |

Table 1 lists the initial molar concentrations of acetic acid and deionized water needed for various molar concentrations of PVA and PVAc.

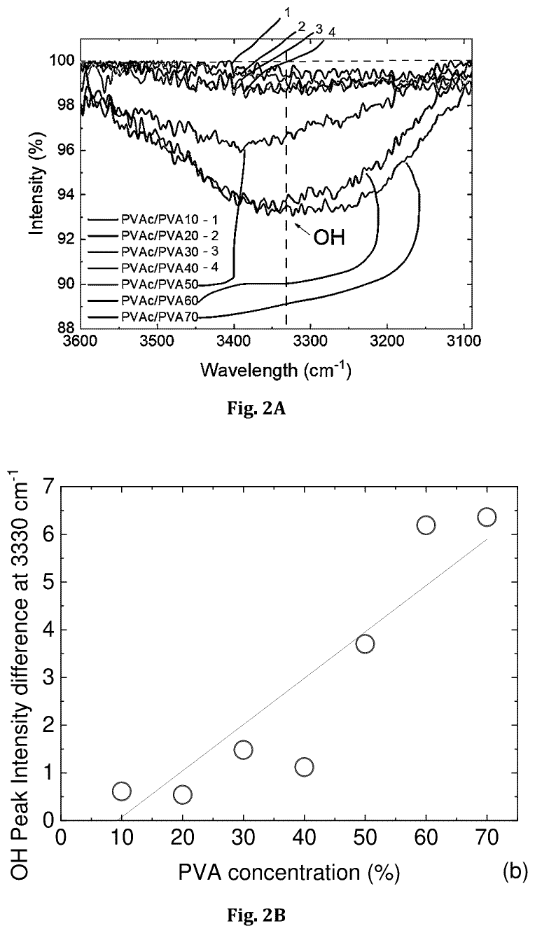

The composition of the PVAc/PVA copolymers was qualitatively examined with Fourier Transform Infrared (FTIR) spectroscopy. The results are shown in FIG. 2A where the FTIR spectra of the copolymers PVAc/PVA10-PVAc/PVA70 are shown. As the theoretical VA concentration increased from 10 mM to 70 mM, the content of hydroxyl groups (at 3330 nm-1) in the resultant copolymer increased (see FIG. 2B). These results indicate that hydrolysis had taken place and confirmed that PVAc/PVA copolymers with different VA concentrations were created. Since the VA concentration was not verified experimentally, the theoretical VA concentrations were calculated using Eq. (3) and therefore, were not a quantitative gauge of the actual VA concentration but a qualitative estimate of the extent of conversion of VAc to VA.

### Example 2

### Preparation of PVAc/Poly(Methyl Vinyl Ether-Co-Maleic Acid) (PMVE-MA) Amphiphilic Copolymers

PVAc/PMVE-MA amphiphilic copolymers consist of PVAc as the hydrophobic component and PMVE-MA as the hydrophilic component.

### Synthesis of PVAc/PMVE-MA Amphiphilic Copolymers

A random PVAc/PMVE-MA copolymer was obtained by further reacting a carboxyl group from the polymer PMVE-MA, . . . . C₇H₈O₄ . . . , (with equal weight to the original PVAc) with a hydroxyl group from the copolymer PVAc/PVA30, . . . . C₆H₁₀O₃ . . . (obtained using the above PVAc hydrolysis procedure) as illustrated by Eq. (4) below and in the schematic in FIG. 1C [28].

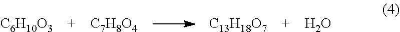

The reaction flask was removed from the water bath and PMVE-MA anhydride (MW 216,000D, Sigma) was added to the flask at room temperature. The flask was heated in a 78±2° C. water bath for 14 min followed by removal from the water bath and left to cool and stored at room temperature until used. Although the final product is referred to as a PVAc/PMVE-MA copolymer, it is actually a copolymer that consists of PVAc and PMVE-MA but also PVA, namely, PVAc/PVA/PMVE-MA. This is because while VA monomers were consumed by the reaction between VA and MVE-MA, additional VA monomers were also created by the ongoing hydrolysis of VAc. The final reaction product is referred to as a PVAc/PMVE-MA copolymer to indicate that the copolymers are a reaction product of PVAc and PMVE-MA as the two starting polymers without excluding either possibility that PVA may be present or absent in the final copolymer.

### Amphiphilic Copolymer Coatings

### Coating Process and Wetting Angle of Coating

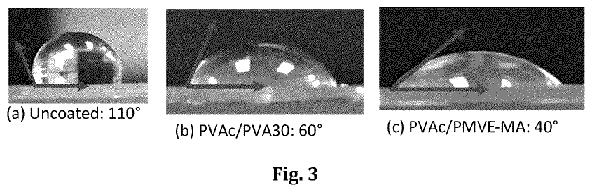

Copolymer coating was done on a model flat silicone surface for ease of experimentation. Each silicone piece was cut to 1 cm by 1 cm. The coating was done in a 10% copolymer solution in a 50-50 ethanol and water mixture. The amphiphilicity of the copolymer was examined with the wetting angle of a 20-μl DI water droplet on the copolymer coating as shown in FIGS. 3A-3C. Silicone is highly hydrophobic with a wetting angle of about 110° as shown in FIG. 3A. Both PVA and PMVE-MA are hydrophilic polymers. Therefore, the coating of PVAc/PVA or PVAc/PMVE-MA on the silicone surface was expected to reduce the wetting angle. Indeed, the PVAc/PVA30 coating reduced the wetting angle from 110° to 60°, as confirmed in FIG. 3B and the coating of PVAc/PMVE-MA further reduced the wetting angle from 110° to 40° (FIG. 3C). These results indicate that the coatings were successful and made the surface more hydrophilic.

### Coating Stability and Durability for at Least 30 Days

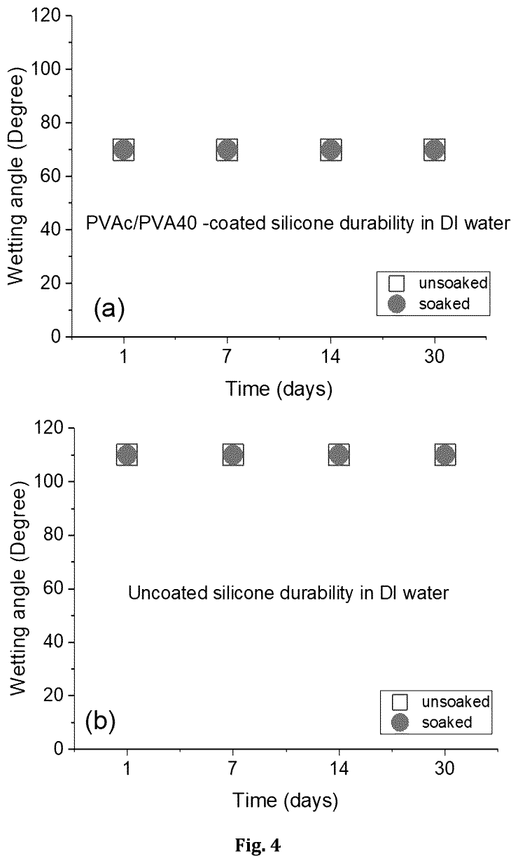

The durability of the coatings was examined by soaking the coated and uncoated silicone pieces in DI water for an extended period (30 days) and taking them out to periodically measure the wetting angle. As an example, the wetting angle versus time of soaked and unsoaked PVAc/PVA40-coated silicone surfaces are shown in FIG. 4A and those of soaked and unsoaked uncoated silicone surfaces are shown in FIG. 4B for comparison. The wetting angle of the soaked and unsoaked PVAc/PVA40-coated surfaces remained at 70° throughout the testing period, which is lower than that of the uncoated silicone surface, indicating the stability and durability of the coating.

### Minimum Inhibitory Concentration (MIC) in Solution

PVAc/PVA and PVAc/PMVE-MA Exhibited μg/Ml MIC for Gram-Positive SA and MRSA

The antimicrobial effects of the aqueous solutions of PVAc/PVA30 (PVAc/PVA thereafter) and PVAc/PMVE-MA copolymers were examined by incubating bacterial cultures at 10⁴ CFU/ml with these copolymer solutions at various concentrations at 37° C. for 2.5 hr. The bacteria studied included Gram-positive bacteria such as Staphylococcus aureus (SA) (ATCC29213), methicillin-resistant Staphylococcus aureus (MRSA) (ATCCbaa1026), and Gram-negative bacteria such as Pseudomonas aeruginosa (PA) (ATCC 27853) and Escherichia coli (EC) K12 (ATCC K12). Following incubation with various concentrations of PVAc/PVA or PVAc/PMVE-MA, and PMVE-MA the cultures were then plated on Agar plates and incubated overnight. The colonies that formed on the agar plates were counted and normalized to the colony count of the control (untreated) suspension and reported as % bacterial viability.

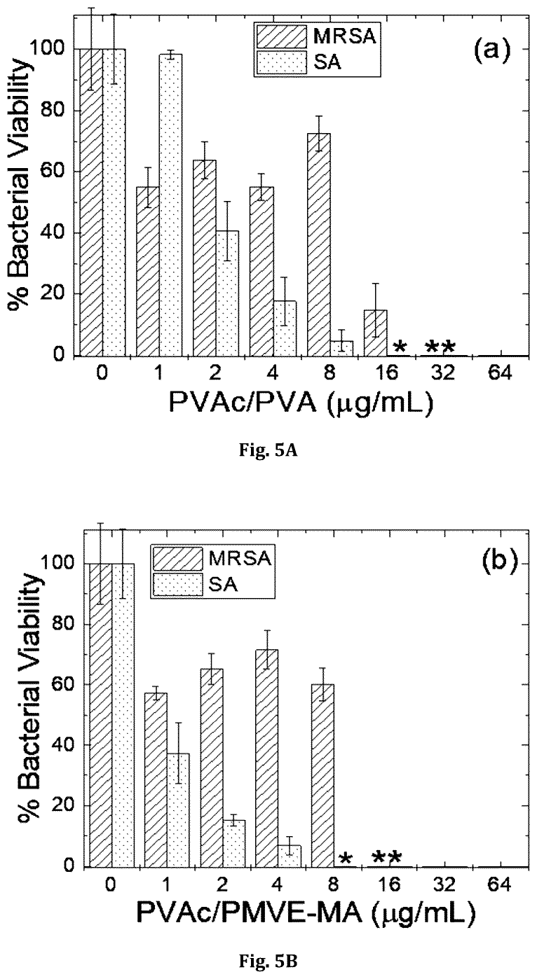

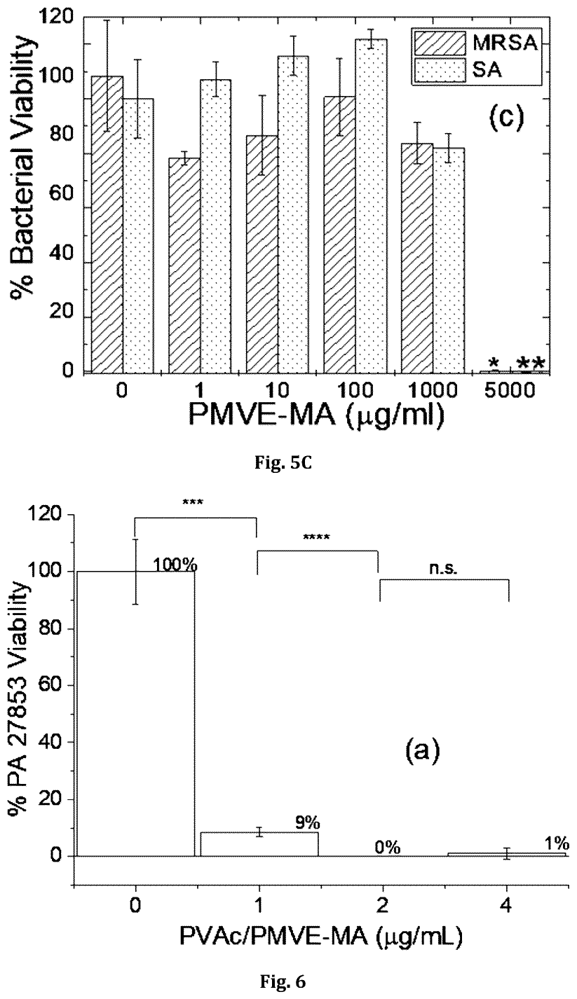

FIGS. 5A-5C show the % bacterial viability of SA (dotted bars) and MRSA (diagonally lined bars) at various concentrations of PVAc/PVA (FIG. 5A), PVAc/PMVE-MA (FIG. 5B), and PMVE-MA (FIG. 5C). PVAc/PVA and PVAc/PMVE-MA exhibited MICs of: 16 μg/ml and 32 μg/ml of PVAc/PVA for SA (*) and MRSA (**), respectively, and MICs of 8 μg/ml and 16 μg/ml of PVAc/PMVE-MA for SA (*) and MRSA (**), respectively while PMVE-MA alone exhibited an MIC exceeding 5000 μg/ml for both SA and MRSA. The measured MICs for SA and MRSA were comparable to those of antibiotics. PVA and PVAc are known to be nonantimicrobial [31-33]. That PVAc/PVA amphiphilic copolymers are antimicrobial and exhibit such low MICs is significant and demonstrates that it is the amphiphilicity of the PVAc/PVA that gave rise to the antimicrobial property and that the antimicrobial capability of PVAc/PVA is far beyond the sum of the antimicrobial capabilities of PVAc and PVA alone. A PVAc/PVA copolymer is non-ionic as both PVAc and PVA carry no charge and thus such copolymers are less likely to disrupt the cellular membrane causing cytotoxicity and injury to tissue and blood vessels, which is an advantage compared to polymers containing, for example, positively charged amine groups.

Although PMVE-MA is the example copolymer used in the US patent No. U.S. Pat. No. 6,403,113 B1 (2002) [34], to illustrate the antimicrobial chemistry claimed in the patent, PMVE-MA was envisioned as an absorbent at 10%-15% [34]. In an antimicrobial hydrogel, the hydrogels also require more than a 10% PVA/PMVE-MA concentration [35]. These concentrations are much higher than the MIC of the current PVAc/PMVE-MA copolymer. The high MIC of PMVE-MA alone was further evidenced by the 5000 μg/ml MIC of PMVE-MA for SA and MRSA in our own study as shown in FIG. 5C. The fact that PVAc/PMVE-MA exhibited MICs of 8 μg/ml and 16 μg/ml for SA and MRSA, respectively, confirms that the high potency of the current amphiphilic copolymer was due to its amphiphilicity and its high molecular weight. PMVE-MA is negatively charged, which is also an advantage as compared to having a positive charge that would bind to and disrupt cells.

PVAc/PMVE-MA Exhibited Low MIC for Gram-Negative PA and E coli K12

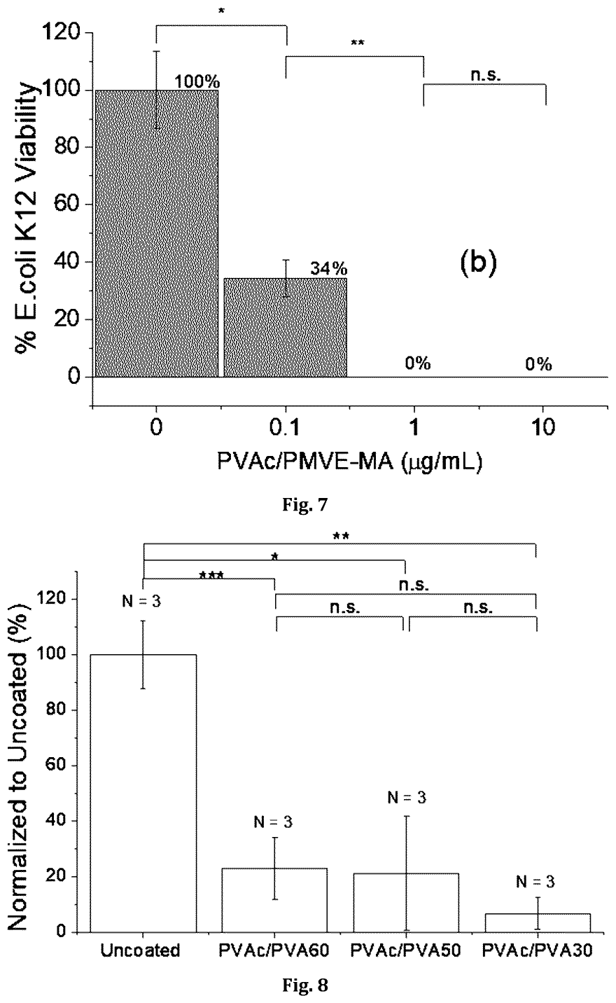

Amphiphilic copolymers are not just antimicrobial but are also effective at concentrations comparable to antibiotics or lower. FIGS. 6 and 7 show % bacterial viability of PA (ATCC 27853 and that of E coli K12 (ATCC 10798) culture incubated at various concentrations of PVAc/PMVE-MA for 2.5 hr, respectively. As can be seen in FIGS. 6 and 7, % bacterial viability of PA diminished at 2 μg/ml of PVAc/PMVE-MA and that of E coli K12 diminished at 1 μg/ml of PVAc/PMVE-MA, suggesting MICs of PVAc/PMVE-MA of 2 μg/ml for PA and 1 μg/ml for E coli K12, respectively, which are again, comparable to the MICs of antibiotics and indicates the excellent antimicrobial effect of the PVAc/PMVE-MA copolymers against Gram-negative bacteria such as PA and E coli K12.

### Antimicrobial Effect of Amphiphilic Coating on Silicone

### PVAc/PVA Coating Reduced % Bacterial Viability in Saline Solution by 93%

Testing of the antimicrobial effect of the coatings was conducted by placing a droplet of bacteria suspension on substrates in either saline solution (4.5 mg/ml of NaCl in DI water) or in broth for 2.5 hr. The bacteria droplets were incubated on uncoated, PVAc/PVA60-coated, PVAc/PVA50-coated, PVAc/PVA30-coated, and PVAc/PMVE-MA-coated silicone surfaces, carefully collected and plated on Agar overnight. The colonies on agar were then counted and normalized to the colony count derived from the bacteria suspension (control). % bacterial viability normalized-to-uncoated for E coli K12 bacteria droplets in saline solution incubated on PVAc/PVA60-, PVAc/PVA50-, and PVAc/PVA30-coated silicone surfaces are shown in FIG. 8. The % bacterial viability was reduced to 23%, 21%, and 6.5% on PVAc/PVA60-, PVAc/PVA50-, and PVAc/PVA30-coated silicone surfaces, respectively, compared to the uncoated silicone surface. Based on statistical testing there was not much difference found between the % bacterial viability of the PVAc/PVA60-, PVAc/PVA50-, and PVAc/PVA30-coated silicone surfaces. However, in comparison to the uncoated silicone surface, all of these coated silicone samples (PVAc/PVA60-, PVAc/PVA50-, PVAc/PVA30-coated silicone) displayed a significant difference in % bacterial viability with p=0.01, p=0.001, and p=0.004, respectively.

### PVAc/PMVE-MA Coating in Saline Further Reduced % Bacterial Viability by 97%

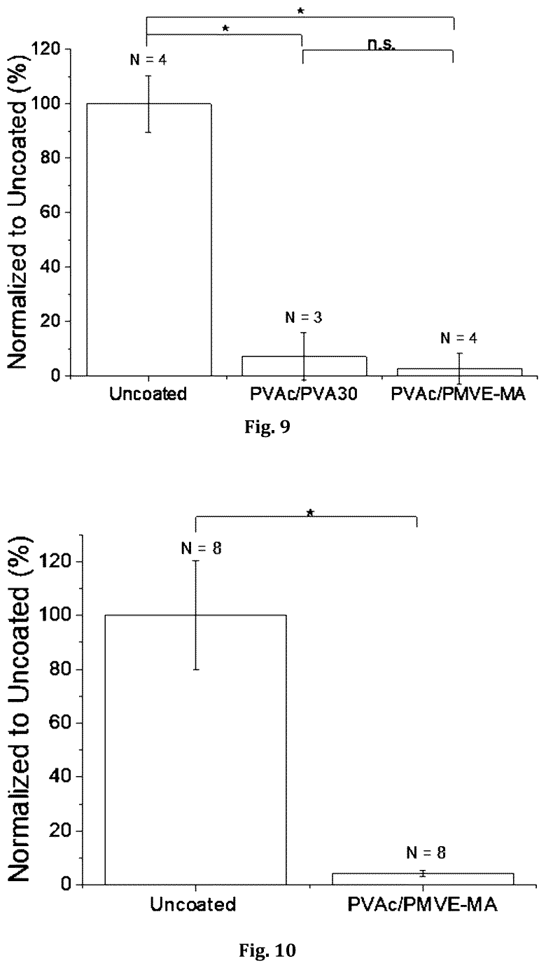

In this study, the % bacterial viability of E coli K12 bacteria droplets in saline solution incubated on PVAc/PVA30-, and PVAc/PMVE-MA-coated silicone surfaces in FIG. 9 was compared to the uncoated silicone surface. The normalized-to-uncoated % bacterial viability was 7.4%, and 2.8% for the PVAc/PVA30-coated, and PVAc/PMVE-MA-coated silicone surfaces, respectively. Based on statistical testing there was no significant difference found between the % bacterial viability of PVAc/PVA30 and PVAc/PMVE-MA, indicating that the PVAc/PVA30 coating had a similar antimicrobial effect as the PVAc/PMVE-MA coating which contains the known antimicrobial agent, PMVE-MA.

### PVAc/PMVE-MA Coating Reduced % Bacterial Viability of PA (ATCC 27853) in Broth by 96%

In this study, the reductions of the % bacterial viability of a PA (ATCC 27853) suspension in broth on uncoated silicone and on a PVAc/PMVE-MA coated silicone were compared. The results are plotted in FIG. 10, which shows that the normalized-to-uncoated % bacterial viability on PVAc/PMVE-MA coating was 4±1%. This shows that the PVAc/PMVE-MA coating reduced the % bacterial viability in broth by about 96±1%, which was within the experimental uncertainty of the result in saline solution of 97%.

### PVAc/PMVE-MA Coating Reduced % Bacterial Viability of SA (ATCC 29213) in Broth by 94%

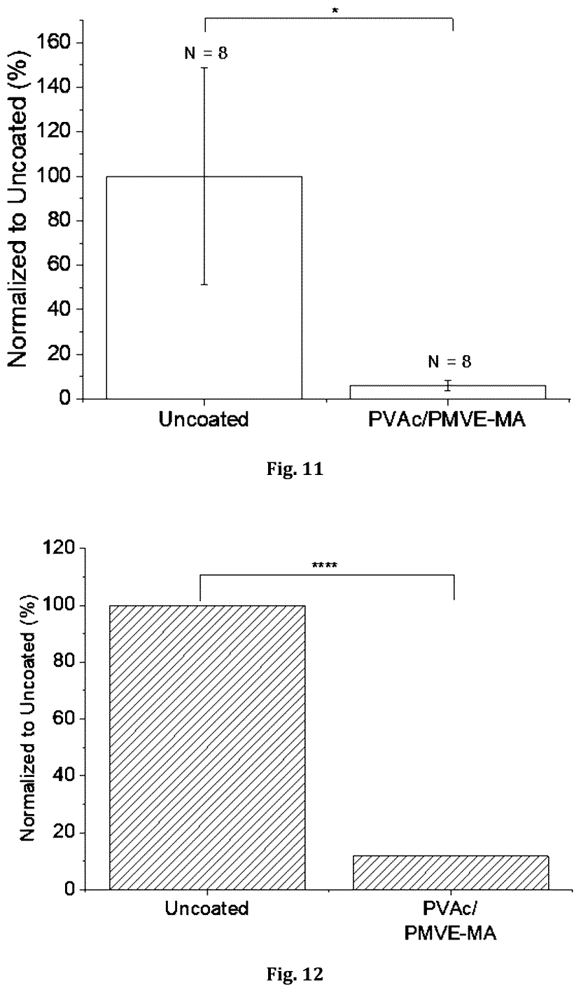

In another study using Staphylococcus aureus (SA) (ATCC 29213) suspended in broth, the % bacterial viability on uncoated and on PVAc/PMVE-MA coated silicone was compared. The normalized-to-uncoated % bacterial viability of SA 29213 suspended in broth on PVAc/PMVE-MA-coated silicone was reduced by 94% compared to uncoated silicone (FIG. 11). Also, in comparison to the % bacterial viability in saline, the % bacterial viability of SA in broth, 6±2%, was within the experimental uncertainty of the measurements.

### PVAc/PMVE-MA Coating Reduced % Bacterial Viability of MRSA (ATCC Baa-1026) by 88%

In a follow-up study, the reduction of % bacterial viability of methicillin-resistant SA (MRSA) (ATCC baa 1026) suspended in broth on uncoated silicone and PVAc/PMVE-MA coated silicone was compared. In FIG. 12, it was demonstrated that the % bacterial viability of MRSA in broth on the PVAc/PMVE-MA coated silicone was 12% (n=8) of the % bacterial viability of the MRSA on the uncoated silicone (n=8). This indicates that the PVAc/PMVE-MA coating on silicone reduced the % bacterial viability for MRSA by 88%, which is comparable to the 94% reduction in % bacterial viability of SA by the same coating. These results support that PVAc/PMVE-MA was equally effective in inhibiting both antibiotic-susceptible and antibiotic-resistant bacteria, a clear advantage in combating antibiotic resistance.

The PVAc/PMVE-MA Coating was Equally Effective with a ≥88% Reduction Against Both Gram-Negative and Gram-Positive Bacteria Including Both Susceptible and Resistant Strains

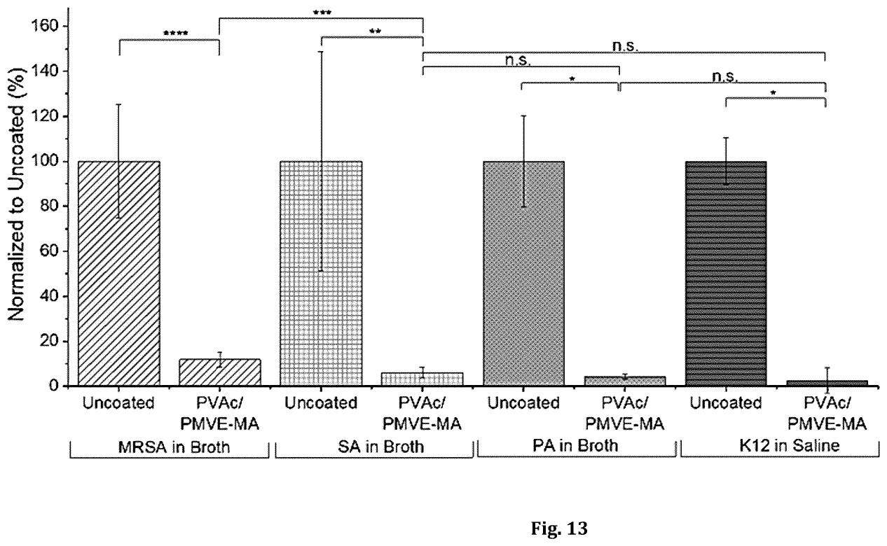

In FIG. 13, a comparison plot of the reduction of % bacterial viability normalized-to-uncoated on a PVAc/PMVE-MA coating with various bacterial suspensions in broth to that of a bacterial suspension in saline solution is shown. As seen in FIG. 13, there was no significant difference in the antibacterial effect of PVAc/PMVE-MA coated silicone on bacteria when it was suspended in broth or saline. This indicates that the PVAc/PMVE-MA coated silicone surface has a similar antimicrobial effect on various bacteria and in the presence or absence of proteins. PA and E coli are Gram-negative, and SA and MRSA are Gram-positive, therefore these results indicate that the PVAc/PMVE-MA coating was equally effective in inhibiting both Gram-positive and Gram-negative bacteria, as well as both antibiotic-susceptible and antibiotic-resistant bacteria.

### Non-Cytotoxicity to Mammalian Cells

Cell line MCF12A cell viability was tested in the presence of PVAc/PMVE-MA and was determined using a broth microdilution assay. In FIG. 14A the % cell viability was shown to decrease with increasing PVAc/PMVE-MA copolymer concentration, with 90% cell viability at the highest concentration evaluated. Although the cell viability dropped to 90% after 2.5 hours of incubation with PVAc/PVME-MA, it may be possible for the viability to increase under longer periods of incubation as the viable cells are allowed to double. In FIG. 14B the % bacterial viability of P. aeruginosa showed zero bacterial viability in the presence of PVAc/PMVE-MA. Comparing to the bacterial viability of PA (ATCC 27853) at the same PVAc/PMVE-MA copolymer concentrations showed the antimicrobial potency of PVAc/PMVE-MA and its potential for biocompatibility at 2-4 μg/mL.

### CONCLUSIONS

The present invention created highly antimicrobial amphiphilic copolymers and coatings that are antimicrobial at microgram/ml concentrations, and which significantly reduced bacterial viability on a silicone surface by more than 96%, both in broth and in saline solution. The coating was also chemically stable and durable, allowing for long-lasting antimicrobial protection against both Gram-positive and Gram-negative bacteria. The coating was equally effective in protecting against antibiotic-susceptible and antibiotic-resistant bacteria. In addition, the antimicrobial amphiphilic copolymers are noncytotoxic. Due to the amphiphilicity, the coating is a monolayer, thin, soft, and non-stick, ideal for minimizing injuries to tissues and blood vessels when inserted.

### REFERENCES

All references cited herein are hereby incorporated by reference in their entirety as if fully set forth herein.
ADDIN EN.REFLIST [1] J. J. Rahal, “Intravascular-catheter-related infections,” Lancet, vol. 351, no. 9117, p. 1739 Jun. 6 1998, doi: 10.1016/S0140-6736 (05) 77782-5.
[2] D. C. McGee and M. K. Gould, “Preventing complications of central venous catheterization,” N Engl J Med, vol. 348, no. 12, pp. 1123-33, Mar. 20 2003, doi: 10.1056/NEJMra011883.
[3] B. W. Trautner, R. A. Hull, and R. O. Darouiche, “Prevention of catheter-associated urinary tract infection,” Curr Opin Infect Dis, vol. 18, no. 1, pp. 37-41, February 2005, doi: 10.1097/00001432-200502000-00007.
[4] Y. Haddadin, P. Annamaraju, and H. Regunath, “Central Line Associated Blood Stream Infections (CLABSI),” StatPearls Publishing, 2017.
[5] Y. Alpay, N. Aykin, P. Korkmaz, H. M. Gulduren, and F. C. Caglan, “Urinary tract infections in the geriatric patients,” Pak J Med Sci, vol. 34, no. 1, pp. 67-72, January-February 2018, doi: 10.12669/pjms.341.14013.
[6] L. E. Nicolle, “Catheter associated urinary tract infections,” Antimicrob Resist Infect Control, vol. 3, p. 23, 2014, doi: 10.1186/2047-2994-3-23.
[7] S. Saint, E. Gaies, K. E. Fowler, M. Harrod, and S. L. Krein, “Introducing a catheter-associated urinary tract infection (CAUTI) prevention guide to patient safety (GPS),” Am J Infect Control, vol. 42, no. 5, pp. 548-50, May 2014, doi: 10.1016/j.ajic.2013.12.019.
[8] S. Saint, J. A. Meddings, D. Calfee, C. P. Kowalski, and S. L. Krein, “Catheter-associated urinary tract infection and the Medicare rule changes,” Ann Intern Med, vol. 150, no. 12, pp. 877-84, Jun. 16 2009, doi: 10.7326/0003-4819-150-12-200906160-00013.
[9] E. P. Magennis, A. L. Hook, P. Williams, and M. R. Alexander, “Making Silicone Rubber Highly Resistant to Bacterial Attachment Using Thiol-ene Grafting,” ACS Appl Mater Interfaces, vol. 8, no. 45, pp. 30780-30787, Nov. 16 2016, doi: 10.1021/acsami.6b10986.
[10] S. Zhang et al., “Enhanced Antibacterial and Antiadhesive Activities of Silver-PTFE Nanocomposite Coating for Urinary Catheters,” ACS Biomater Sci Eng, vol. 5, no. 6, pp. 2804-2814 Jun. 10 2019, doi: 10.1021/acsbiomaterials.9b00071.
[11] M. Al-Qahtania, A. Safanb, G. Jassimb, and S. Abadla, “Efficacy of anti-microbial catheters in preventing catheter associated urinary tract infections in hospitalized patients: A review on recent updates,” Journal of Infection and Public Health, vol. 12, 2019.
[12] K. G. Neoh, M. Li, E. T. Kang, E. Chiong, and P. A. Tambyah, “Surface modification strategies for combating catheter-related complications: recent advances and challenges,” J Mater Chem B, vol. 5, no. 11, pp. 2045-2067 Mar. 21 2017, doi: 10.1039/c6tb03280j.
[13] D. G. Desai, K. S. Liao, M. E. Cevallos, and B. W. Trautner, “Silver or nitrofurazone impregnation of urinary catheters has a minimal effect on uropathogen adherence,” J Urol, vol. 184, no. 6, pp. 2565-71, December 2010, doi: 10.1016/j.juro.2010.07.036.
[14] J. R. Johnson, M. A. Kuskowski, and T. J. Wilt, “Systematic review: antimicrobial urinary catheters to prevent catheter-associated urinary tract infection in hospitalized patients,” Ann Intern Med, vol. 144, no. 2, pp. 116-26, Jan. 17 2006, doi: 10.7326/0003-4819-144-2-200601170-00009.
[15] M. L. W. Knetsch and L. H. Koole, “New Strategies in the Development of Antimicrobial Coatings: The Example of Increasing Usage of Silver and Silver Nanoparticles,” Polymers, vol. 3, 2011.
[16] K. N. J. Stevens et al., “The relationship between the antimicrobial effect of catheter coatings containing silver nanoparticles and the coagulation of contacting blood,” Biomaterials, vol. 30, 2009.
[17] S. Zanini, A. Polissi, E. A. Maccagni, E. C. Dell′Orto, C. Liberatore, and C. Riccardi, “Development of antibacterial quaternary ammonium silane coatings on polyurethane catheters,” J Colloid Interface Sci, vol. 451, pp. 78-84, Aug. 1 2015, doi: 10.1016/j.jcis.2015.04.007.
[18] C. Monteiro, F. Costa, A. M. Pirttila, M. V. Tejesvi, and M. C. L. Martins, “Prevention of urinary catheter-associated infections by coating antimicrobial peptides from crowberry endophytes,” Sci Rep, vol. 9, no. 1, p. 10753, Jul. 24 2019, doi: 10.1038/s41598-019-47108-5.
[19] J. L. Low et al., “Development of a polymer-based antimicobial coating for efficacious urinary catheter protection,” Biotechnology Notes, vol. 2, 2020.
[20] R. L. Shum, S. R. Liu, A. Caschera, and D. A. Foucher, “UV-Curable Surface-Attached Antimicrobial Polymeric Onium Coatings: Designing Effective, Solvent-Resistant Coatings for Plastic Surfaces,” ACS Appl Bio Mater, vol. 3, no. 7, pp. 4302-4315 Jul. 20 2020, doi: 10.1021/acsabm.0c00359.
S. Vasudevan, R. D. Durai, D. R. Chellappan, V. H. B. Narayanan, P. C. Prabu, and A. P. Solomon, “A polymer-based anti-quorum catheter coating to challenge MDR Staphylococcus aureus: in vivo and in vitro approaches,” J Antimicrob Chemother, vol. 74, no. 6, pp. 1618-1626 Jun. 1 2019, doi: 10.1093/jac/dkz094.
[22] D. L. Williams et al., “In vivo efficacy of a silicone-cationic steroid antimicrobial coating to prevent implant-related infection,” Biomaterials, vol. 33, 2012.
[23] G. Xu, P. Liu, D. Pranantyo, L. Xu, K. G. Neoh, and E. T. Kang, “Antifouling and Antimicrobial Coatings from Zwitterionic and Cationic Binary Polymer Brushes Assembled via “Click” Reactions,” Industrial & Engineering Chemistry Research, 2017.
[24] A. Chen et al., “Antimicrobial Anilinium Polymers: The Properties of Poly(N,N-dimethylaminophenylene methacrylamide) in Solution and as Coatings,” JOURNAL OF POLYMER SCIENCE, PART A: POLYMER CHEMISTRY, 2019.
[25] Y. Yang, Z. Cai, Z. Huang, X. Tang, and X. Zhang, “Antimicrobial cationic polymers: from structural design to functional control,” Polymer Journal, vol. 50, 2018.
[26] K. S. Huang, C. H. Yang, S. L. Huang, C. Y. Chen, Y. Y. Lu, and Y. S. Lin, “Recent Advances in Antimicrobial Polymers: A Mini-Review,” International Journal of Molecular Sciences, vol. 17, 2016.
[27] C. Ergene and E. F. Palermo, “Antimicrobial Synthetic Polymers: An Update on Structure-Activity Relationships,” Curr Pharm Des, vol. 24, no. 8, pp. 855-865, 2018, doi: 10.2174/1381612824666180213140732.
[28] S. Bandyopadhyay et al., “Slippery liquid infused fluoropolymer coating for central lines to reduce catheter associated clotting and infections,” Sci Rep, vol. 10, no. 1, p. 14973, Sep. 11 2020, doi: 10.1038/s41598-020-71711-6.
[29] X. Guo, F. Deng, L. Li, and R. K. Prud′homme, “Synthesis of biocompatible polymeric hydrogels with tunable adhesion to both hydrophobic and hydrophilic surfaces,” Biomacromolecules, vol. 9, no. 6, pp. 1637-42, June 2008, doi: 10.1021/bm800142z.
[30] S. Park and H. Yoon, “Acid-Catalyzed Hydrolysis Reaction of Poly(vinyl acetate),” Polymer (Korea), vol. 29, p. 4, 2005.
[31] D. Hu and L. Wang, “Physical and antibacterial properties of polyvinyl alcohol films reinforced with quaternized cellulose,” Journal of Applied Polymer Science, vol. 133, no. 25, Jun. 5, 2016 2016.
[32] L. Mitrea et al., “Poly(vinyl alcohol)-Based Biofilms Plasticized with Polyols and Colored with Pigments Extracted from Tomato By-Products,” Polymers (Basel), vol. 12, no. 3, Mar. 2 2020, doi: 10.3390/polym12030532.
[33] K. Szabo et al., “Active Packaging-Poly(vinyl alcohol) films enriched with Tomato by-products extract,” Coatings, vol. 10, 2020, doi: 10.3390.
[34] I. Corzani, “ANTI-MICROBICAGENT,” U.S. Pat. No. 10,197,565 B2, 2002.
[35] E. Calo, J. M. S. de Barros, M. Fernandez-Gutierrez, J. San Roman, B. L., and V. V. Khutoryanskiy, “Antimicrobial hydrogels based on autoclaved poly(vinyl alcohol) and poly(methyl vinyl ether-altmaleic anhydride) mixtures for wound care applications,” RSC Adv., vol. 6, p. 9, 2016, doi: 10.1039/c6ra08234c.
[36] Mushtaq, Shehla, et al. “Antibacterial Amphiphilic Copolymers of Dimethylamino Ethyl Methacrylate and Methyl Methacrylate to Control Biofilm Adhesion for Antifouling Applications.” Polymers, vol. 13, no. 2, 2021, p. 216., https://doi.org/10.3390/polym13020216.
[37] Chang, Chih-Hao, et al. “Quaternized Amphiphilic Block Copolymers as Antimicrobial Agents.” Polymers, vol. 14, no. 2, 8 Jan. 2022, p. 250, www.ncbi.nlm.nih.gov/pmc/articles/PMC8781064/, https://doi.org/10.3390/polym14020250. Accessed 31 Oct. 2022.

What is claimed is:

1. An antimicrobial amphiphilic copolymer comprising units derived from one or more hydrophobic polymers and one or more hydrophilic polymers,
wherein the copolymer is selected from the group consisting of non-ionic and anionic copolymers and the copolymer is a random copolymer or a block copolymer.

2. The antimicrobial amphiphilic copolymer of claim 1, wherein the hydrophobic polymer is selected from the group consisting of poly-vinyl acetate and poly(maleic anhydride-alt-1-octadecene); and
the hydrophilic polymer is selected from the group consisting of negatively-charged polymers and polymers with no charges.

3. The antimicrobial amphiphilic copolymer of claim 2, wherein the hydrophilic polymer is selected from the group consisting of poly-(methyl vinyl ether)-maleic anhydride, polyvinyl-alcohol, polyethylene glycol, polysaccharide, any combination thereof, and any polymer containing a hydroxyl group.

4. The antimicrobial amphiphilic copolymer of claim 1, wherein the hydrophobic polymer has a molecular weight of from about 20 D to about 10,000,000 D, as measured by gel permeation chromatography, the hydrophilic polymer has a molecular weight of from about 20 D to about 10,000,000 D, as measured by gel permeation chromatography and the copolymer has a molar ratio of the hydrophobic polymer to the hydrophilic polymer of from about 0.0001 to 10000.

5. The antimicrobial amphiphilic copolymer of claim 1, wherein the hydrophobic polymer is poly-vinyl acetate and the hydrophilic polymer is selected from the group consisting of polyethylene glycol, polyvinyl-alcohol and poly-(methyl vinyl ether)-maleic anhydride.

6. The antimicrobial amphiphilic copolymer of claim 1, wherein the hydrophobic polymer is poly(maleic anhydride-alt-1-octadecene) and the hydrophilic polymer is polyethylene glycol.

7. The antimicrobial amphiphilic copolymer of claim 1, wherein the hydrophilic polymer is a hydroxyl group containing hydrophilic polymer selected from the group consisting of poly(vinyl alcohol), glycerol propoxylate, poly(vinyl alcohol co-ethylene, poly(2-hydroxyethyl methacrylate), α,ω-bis(hydroxy)-terminated poly(ethylene glycol), α,ω-bis(hydroxy)-terminated poly(ethylene oxide), α-dibenzylmethylene-terminated pooly(ethylene glycol), poly(ethylene glycol) decyl ether, poly(ethylene glycol)ethyl ether, poly(ethylene glycol) and combinations of two or more of these hydrophilic polymers.

8. The antimicrobial amphiphilic copolymer of claim 1, wherein the hydrophobic polymer is a carboxyl group containing hydrophobic polymer selected from the group consisting of poly(acrylic acid), poly(ethylene-alt-maleic anhydride), poly(4-styrenesulfonic acid-co-maleic acid), poly(methyl vinyl ether-alt-maleic anhydride), poly(methyl vinyl ether-alt-maleic acid, poly(styrene-alt-maleic acid), poly(methyl vinyl ether-alt-maleic acid monoethyl ester), poly(2-propylacrylic acid), poly(2-ethlacrylic acid), poly(α-ethylacrylic acid), poly(methacrylic acid), poly(α-propylacrylic acid) and combinations of two or more of these hydrophilic polymers.

9. The antimicrobial amphiphilic copolymer of claim 1, wherein the hydrophobic polymer is selected from the group consisting of poly(vinyl acetate), poly(ethylene-vinyl acetate) and combinations thereof.

10. The antimicrobial amphiphilic copolymer of claim 1, wherein the copolymer is devoid of amine functionalities or any positively charged functionalities.

11. The antimicrobial amphiphilic copolymer of claim 1, wherein the minimum inhibitory concentration of the copolymers for methicillin-resistant Staphylococcus aureus and Staphylococcus aureus is from about 0.01 μg/ml-1000 μg/ml and/or the copolymer can inhibit both Gram-positive and Gram-negative bacteria with minimum inhibitory concentration in a range of from about 0.01 μg/ml-1000 μg/ml, and/or the copolymer can inhibit both antibiotic-susceptible and antibiotic-resistant bacteria with a minimum inhibitory concentration in a range of from about 0.01 μg/ml-1000 μg/ml, all as determined by a broth microdilution assay.

12. A method for preparing the antimicrobial amphiphilic copolymer of claim 5, comprising a step of partially hydrolyzing a PVAc polymer using an acid-catalyzed hydrolysis reaction to form a poly-vinyl acetate-polyvinyl-alcohol (PVAc/PVA) random copolymer comprising a vinyl acetate-vinyl alcohol (VAc/VA) segment of the copolymer according to Formula I:

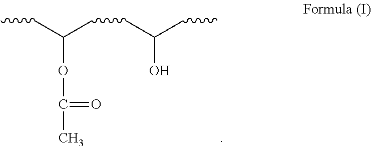

13. A method of preparing the antimicrobial amphiphilic copolymer of claim 5, comprising:
partially hydrolyzing a polyvinyl acetate polymer using an acid-catalyzed hydrolysis reaction to form a poly-vinyl acetate-polyvinyl-alcohol random copolymer, and
reacting the poly-vinyl acetate-polyvinyl-alcohol random copolymer with poly(methyl vinyl ether-co-maleic acid) via a condensation reaction to form a polyvinyl acetate poly(methyl vinyl ether-co-maleic acid copolymer comprising a vinyl acetate-methyl vinyl ether-co-maleic acid segment of the copolymer according to Formula II:

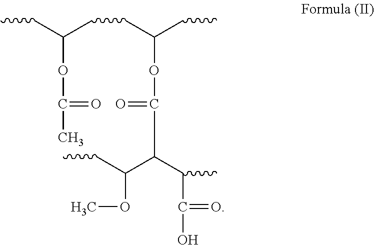

14. A method of preparing the antimicrobial amphiphilic copolymer of claim 6, comprising reacting poly(maleic anhydride-alt-1-octadecene) and polyethylene via a condensation reaction to form a poly(maleic anhydride-alt-1-octadecene)-polyethylene glycol random copolymer.

15. A method of preparing the antimicrobial amphiphilic copolymer of claim 1, comprising steps of:
partially hydrolyzing a PVAc polymer using an acid-catalyzed hydrolysis reaction to form a poly-vinyl acetate-polyvinyl-alcohol (PVAc/PVA) random copolymer, and
reacting the polyvinyl acetate-polyvinyl-alcohol random copolymer with polyethylene glycol (PEG) to form a polyvinyl acetate-polyethylene glycol (PVAc/PEG) random copolymer.

16. An antimicrobial amphiphilic copolymer coating consisting of a monolayer coating comprising the antimicrobial amphiphilic copolymer of claim 1, wherein the hydrophobic component of the copolymer facilitates binding of the copolymer to a surface and the hydrophilic part forms an outer surface.

17. The coating of claim 16, wherein the coating when applied to a hydrophobic surface is configured to modify a wetting angle of the hydrophobic surface from above 110° to below 40°.

18. The coating of claim 16, having a thickness of from about 1 nm to 10,000 nm.

19. A catheter comprising the coating of claim 16 on one or both of an interior and an exterior surface of the catheter and wherein the coating is nontoxic to human cells in a minimum inhibitory concentration range of the copolymer of from about 0.0001 μg/ml-10000 μg/ml.
